(19) United States
(12) Patent Application Publication
(10) Pub. No.: US 2017/0182210 A1
(43) Pub. Date: Jun. 29, 2017

(54) TISSUE ENGINEERING SCAFFOLD MATERIAL AND BIODEGRADABLE POLYESTER ELASTOMER THEREOF

(71) Applicant: NATIONAL TSING HUA UNIVERSITY, Hsinchu City (TW)

(72) Inventors: JANE WANG, LEXINGTON, MA (US); KEN-SEN CHOU, HSINCHU CITY (TW); SUNG-NIEN HSU, HSINCHU CITY (TW)

(21) Appl. No.: 15/154,263

(22) Filed: May 13, 2016

(30) Foreign Application Priority Data
Dec. 25, 2015 (TW) 104143780

(51) Int. Cl. A61L 27/18; A61L 27/58; C08G 63/84; C08G 63/16; C08G 63/85; C08G 63/82

(52) U.S. Cl. CPC A61L 27/18; C08G 63/85; C08G 63/826; C08G 63/84; C08G 63/16; A61L 27/58; A61L 2400/18

(57) ABSTRACT

Disclosed is a biodegradable polyester elastomer which is formed by an esterification reaction between an acidic compound and an alcohol compound using a superacidic catalyst. Thereby, the reaction time of the esterification reaction can be reduced remarkably.

### BACKGROUND OF THE INVENTION

1. Field of the Invention

The instant disclosure relates to a biomedical material; in particular, to a biodegradable polyester elastomer with remarkable biological compatibility, mechanical properties, and biodegradability and a tissue engineering scaffold material using the same. 2. Description of Related Art

In recent years, the incidence of ischaemic vascular diseases has increased year by year due to bad eating habits and aging population. In addition, cerebral, cardiac, or peripheral vascular disease caused by artery ischemia seriously affects human health, and thereby has more readily attracted the attention of doctors and researchers. Methods of clinical treatment for ischaemic diseases include targeting drugs, cavity intervention, vascular bridges, etc. However, these methods have the following problems: (1) the need for secondary surgery due to postoperative vascular occlusion; (2) the lack of biological compatibility and mechanical properties of intervention materials; and (3) the lack of stability and therapeutic effect of drugs. Hence, there is a need for methods of promoting vascularization of ischaemic tissue to effectively recover blood supply.

Vascular tissue engineering is one of the most effective ways to solve the aforementioned problems, and how to choose the best tissue engineering scaffold material is the key point thereof. In general, a suitable tissue engineering scaffold material should have the advantages of high specific surface area, good channel connectivity, high biological compatibility, adjustable degradation rate, good mechanical properties, and environmental benefits of cell culture and tissue growth. A material with high biological compatibility usually means that said material is a low toxicity non-carcinogenic material, and cannot cause any allergic reaction, thrombolysis, tissue multiplication, and infection.

Since tissue engineering scaffold materials with the characteristics of mechanical stimulation and biodegradability are beneficial to vascularization of ischaemic tissue, biodegradable elastomers are widely used in the field of biomedical materials. Biodegradable elastomers can be thermoplastic and thermoset. The thermoplastic elastomer is a block polymer which includes soft segments and hard segments interacting with the soft segments, and the thermoset elastomer is a star-branched polymer. Yadong Wang et al. disclose a melt-polymerization reaction between sebacic and glycerol (1:1 molar ration) for the production of poly(glycerol sebacate) (PGS). However, said melt-polymerization reaction will not only result in bad performance, but also take 24 hours or more. There is an urgent need for technologies used to improve the reaction rate and product yields of polymerization of biodegradable elastomers.

### SUMMARY OF THE INVENTION

The object of the instant disclosure is to provide a biodegradable polyester elastomer with high production efficiency and overall performance.

In order to achieve the aforementioned objects, according to an embodiment of the instant disclosure, the biodegradable polyester elastomer is characterized in that it is formed by an esterification reaction between an acidic compound and an alcohol compound with a superacidic catalyst.

Preferably, the superacidic catalyst is selected from zirconium oxides with sulfate ion (ZrO₂/SO₄²⁻), titanium oxides with sulfate ion (TiO₂/SO₄²⁻), stannum oxides with sulfate ion (SnO₂/SO₄²⁻), hafnium oxides with sulfate ion (HfO₂/SO₄²⁻), iron oxides with sulfate ion (Fe₂O₃/SO₄²⁻), aluminum oxides with sulfate ion (Al₂O₃/SO₄²⁻), and any combination thereof.

Preferably, the superacidic catalyst is in an amount between 0.01 to 1.00 wt % based on 100 wt % of the total weight of the acidic compound and the alcohol compound.

Preferably, the acidic compound is selected from polybasic acids having more than 6 carbon atoms, the alcohol compound is selected from polyhydric alcohols having 4 to 10 carbon atoms, dihydric alcohols having 4 to 10 carbon atoms, and any combination thereof.

Preferably, the molar ratio of the alcohol compound to the acidic compound is 2:1 or greater than 2:1.

Preferably, the acidic compound is sebacic acid, and the alcohol compound is glycerol.

Preferably, the esterification reaction is carried out at a temperature in the range of 120 to 170° C.

Preferably, the esterification reaction is carried out at a vacuum pressure in the range of 300 to 500 mTorrs.

Preferably, the esterification reaction is carried out under a reaction pressure less than 760 mTorrs.

Another object of the instant disclosure is to provide a tissue engineering scaffold material which comprises the aforementioned biodegradable polyester elastomer.

Base on the above, by using the superacidic catalyst to promote the esterification reaction, especially to the esterification reaction of PGS elastomer, the reaction time can be reduced to 60 minutes or lower. The resulting biodegradable polyester elastomer has remarkable biological compatibility, mechanical properties, and biodegradability.

In order to further appreciate the characteristics and technical contents of the instant disclosure, references are hereunder made to the detailed descriptions and appended drawings in connection with the instant disclosure. However, the appended drawings are merely shown for exemplary purposes, rather than being used to restrict the scope of the instant disclosure.

### BRIEF DESCRIPTION OF THE DRAWINGS

FIG. 1 is a flow diagram illustrating a manufacturing method according to a preferred embodiment of the present invention;

FIG. 2 is a schematic diagram illustrating an esterification reaction between an acidic compound and an alcohol compound without adding any superacidic catalyst; and

FIGS. 3A and 3B are schematic diagrams illustrating another esterification reaction between an acidic compound and an alcohol compound with a superacidic catalyst.

### DETAILED DESCRIPTION OF THE PREFERRED EMBODIMENTS

Biodegradable elastomers typically experience mechanical forces and deformation in daily use, and do not appear to affect surrounding tissues during the process of recovery. In addition, biodegradable elastomers with high water absorption capability, high hydrophilicity, and good mechanical properties like proteins can be applied in the biomedical field to be used as the template for formation of tissue engineering scaffold materials. Hence, the present invention provides a biodegradable polyester elastomer which is a prepolymer formed by an esterification reaction under specific reaction conditions (e.g. temperature, pressure, vacuum level) using a superacidic catalyst. In this way, the reaction time can be reduced to 60 minutes or lower to meet the requirements of large-scale production. Most important of all, the superacidic catalyst can be recovered from the prepolymer for reuse.

The aforementioned illustrations and following detailed descriptions are exemplary for the purpose of further explaining the scope of the instant disclosure. Other objectives and advantages related to the instant disclosure will be illustrated in the subsequent descriptions and appended drawings.

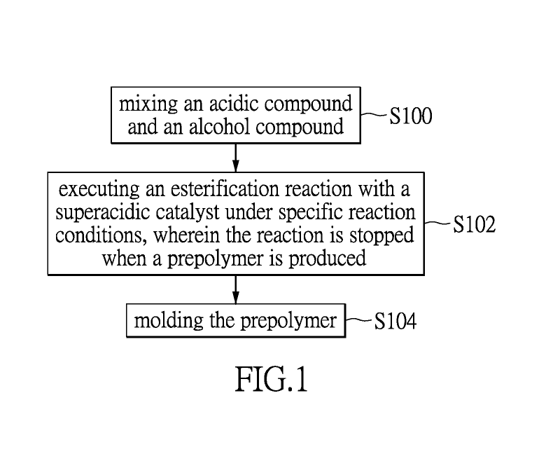

Please refer to FIG. 1. FIG. 1 is a flow diagram illustrating a manufacturing method according to a preferred embodiment of the present invention. The manufacturing method includes a step S100 of mixing an acidic compound and an alcohol compound, a step S102 of executing an esterification reaction with a superacidic catalyst under specific reaction conditions, wherein the reaction is stopped when a prepolymer is produced, and a step S104 of molding the prepolymer.

It is worth mentioning that, by using the superacidic catalyst to promote the esterification reaction, the reaction time can be reduced remarkably, and the comprehensive properties of the instant polyester elastomer (e.g. PGS) can be ensured. Therefore, the instant polyester elastomer has a very wide application in the biomedical field.

In the step S100, the acidic compound and the alcohol compound are mixed in a reactor. The acidic compound is selected from polybasic acids such as polybasic acids having more than 6 carbon atoms. Specific examples of the polybasic acids having more than 6 carbon atoms include: adipic acid, suberic acid, azelaic acid, citric acid, phthalic acid, isophthalic acid, trimellitic acid, 1,2,4,5-pyromellitic acid, etc. Please note that any suitable reactor known in the art can be used to mix the acidic compound and the alcohol compound.

The alcohol compound is selected from polyhydric alcohols such as polyhydric alcohols having 4 to 10 carbon atoms. Specific examples of the polyhydric alcohols having 4 to 10 carbon atoms include: ethylene glycol, 1,2-propylene glycol, 1,3-propane diol, glycerol, 1,4-butane diol, 1,3-butane diol, 1,6-hexane diol, 1,10-decane diol, diethylene glycol, triethylene glycol, pentaerythritol, pentaerythritol, etc. Preferably, the acidic compound is sebacic acid, and the alcohol compound is glycerol. The molar ratio of the alcohol compound to the acidic compound is 2:1 or greater than 2:1.

In the step S102, the superacidic catalyst is added to the reaction mixture of the acidic compound and the alcohol compound in solid form. The superacidic catalyst can serve as both the reaction starting and sustaining catalyst. Preferably, the esterification reaction is carried out at a temperature in the range of 120 to 170° C. at a vacuum pressure in the range of 300 to 500 mTorrs under a reaction pressure less than 760 mTorrs. Thereby the reaction time can be shortened to 1-2 hours. However, a reaction pressure more than 760 mTorrs may result in longer reaction time of 10-12 hours.

For the instant embodiment, the superacidic catalyst is a solid superacidic catalyst with sulfate ion (e.g. sulfated metal oxides, SMOs) as shown in the formula (1) or (2), where M represents metal such as Zr, Ti, Sn, Hf, Fe, Al.

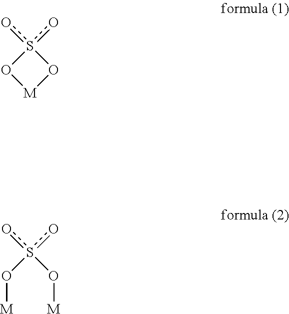

It is worth to note that, superacidic catalysts have a tetragonal crystal structure and exhibits Lewis and Brønsted acid properties, and thus can promote the esterification reaction between an acidic compound and an alcohol compound. Moreover, the superacidic catalysts have good high temperature stability, and can be easily synthesized and reproduced. In addition, the process for manufacturing superacidic catalysts is very simple, and is suitable for industrial production.

Specific examples of the superacidic catalyst include: zirconium oxides with sulfate ion (ZrO₂/SO₄²⁻), titanium oxides with sulfate ion (TiO₂/SO₄²⁻), stannum oxides with sulfate ion (SnO₂/SO₄²⁻), hafnium oxides with sulfate ion (HfO₂/SO₄²⁻), iron oxides with sulfate ion (Fe₂O₃/SO₄²⁻), aluminum oxides with sulfate ion (Al₂O₃/SO₄²⁻), etc. Preferably, the superacidic catalyst is titanium oxides with sulfate ion (TiO₂/SO₄²⁻), and is in an amount between 0.01 to 1.00 wt % based on 100 wt % of the total weight of the acidic compound and the alcohol compound. The reason is that the esterification reaction with a catalyst content of less than 0 01 wt % may result in low yield.

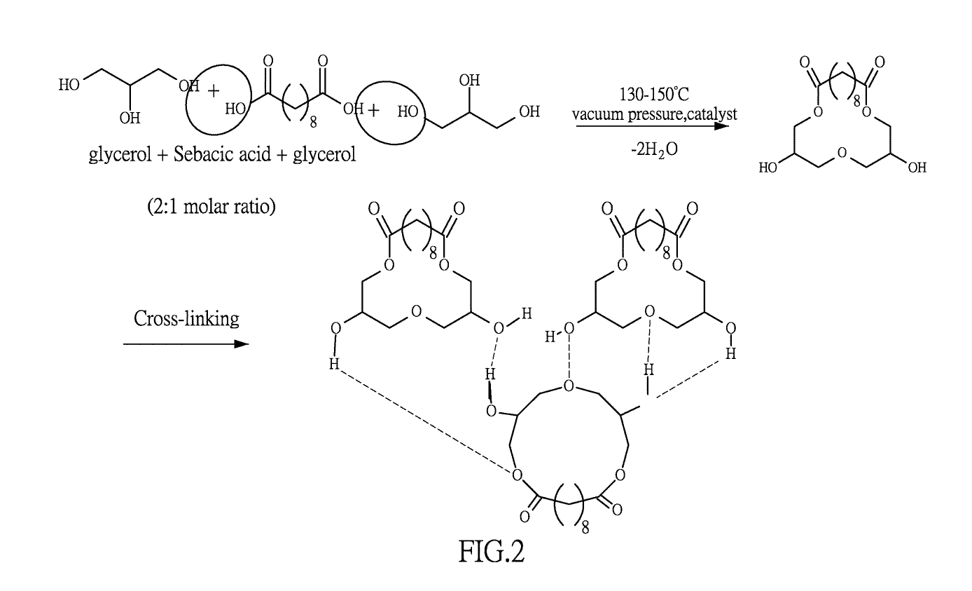

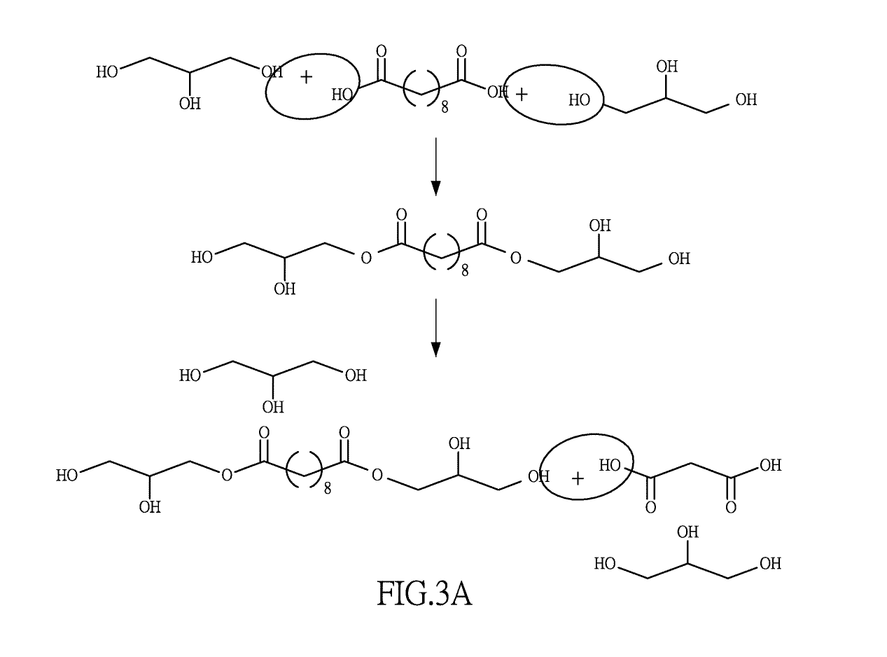

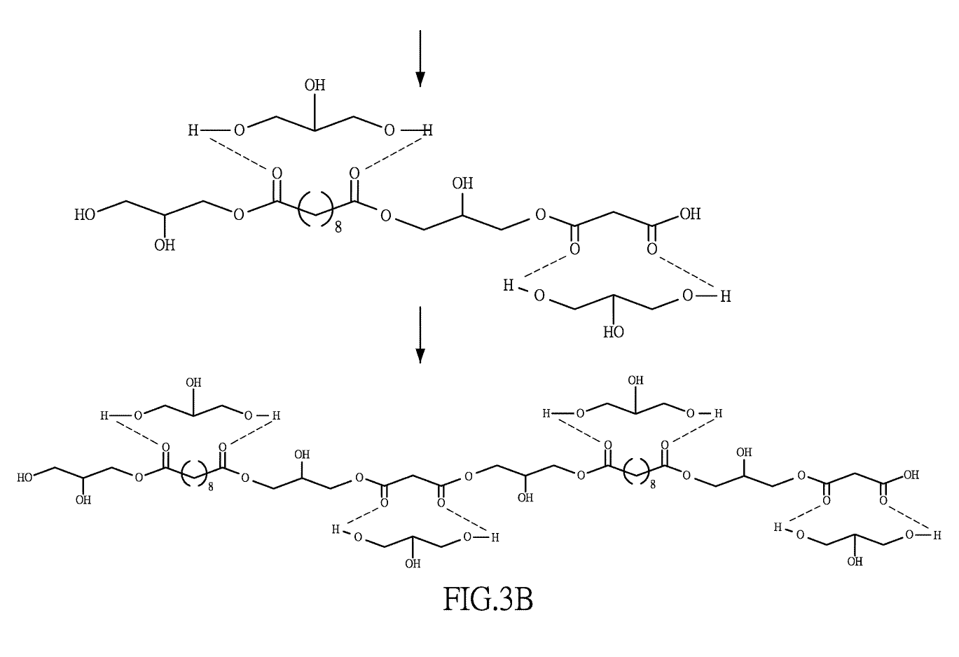

Please refer to FIGS. 2, 3A, and 3B. Considering the properties of superacidic catalysts mentioned above, the following will describe two different reaction mechanisms associated with an esterification reaction of sebacic acid and glycerol with or without a superacidic catalyst. As shown in FIG. 2, an esterification reaction is carried out at a temperature in the range of 130 to 150° C. without adding any superacidic catalyst while the molar ratio of sebacic acid to glycerol is 1:2. In the formation of poly(glycerol sebacate) (PGS), two OH— radicals separate from two ends of the main chain of sebacic acid respectively, a hydrogen radical separates from one end of the main chain of glycerol, and thus the main chain of sebacic acid is linked to two main chains of glycerol. The resulting copolymers are directly cross-linked together to form a cyclic product without catalyst.

As shown in FIGS. 3A and 3B, another esterification reaction of sebacic acid and a stoichiometric excess of glycerol is carried out at a temperature in the range of 130 to 150° C. with a superacidic catalyst of 1 wt %. Please note that after the formation of PGS, hydrogen radicals will separate from one end or two ends of any one main chain of the stoichiometric excess of glycerol with the superacidic catalyst, and thus a plurality of the main chains of the stoichiometric excess of glycerol will be linked to the resulting copolymers continuously to prepare a highly elastic linear product (prepolymer).

Finally, in the step S102, the prepolymer having a cross-linking agent dispersed therein can be molded to have a regular shape. The finished article has remarkably good mechanical property (e.g. tensile strength), wear resistance, solvent resistance, weather resistance, and gas tightness. Please note that any suitable molding means known in the art can be used to shape the prepolymer.

To sum up, by using the superacidic catalyst to promote the esterification reaction, especially to the esterification reaction of PGS elastomer, the reaction time can be reduced to 60 minutes or lower. The resulting biodegradable polyester elastomer has remarkable biological compatibility, mechanical property, and biodegradability. Therefore, the instant polyester elastomer has a very wide application in the biomedical field.

Moreover, the superacidic catalyst can be recovered from the prepolymer for reuse.

The descriptions illustrated supra set forth simply the preferred embodiments of the instant disclosure; however, the characteristics of the instant disclosure are by no means restricted thereto. All changes, alterations, or modifications conveniently considered by those skilled in the art are deemed to be encompassed within the scope of the instant disclosure delineated by the following claims.

What is claimed is:

1. A biodegradable polyester elastomer characterized in that it is formed by an esterification reaction between an acidic compound and an alcohol compound with a superacidic catalyst.

2. The biodegradable polyester elastomer according to claim 1, wherein the superacidic catalyst is selected from zirconium oxides with sulfate ion (ZrO₂/SO₄²⁻), titanium oxides with sulfate ion (TiO₂/SO₄²⁻), stannum oxides with sulfate ion (SnO₂/SO₄²⁻), hafnium oxides with sulfate ion (HfO₂/SO₄²⁻), iron oxides with sulfate ion (Fe₂O₃/SO₄²⁻), aluminum oxides with sulfate ion (Al₂O₃/SO₄²⁻), and any combination thereof.

3. The biodegradable polyester elastomer according to claim 1, wherein the superacidic catalyst is in an amount between 0.01 to 1.00 wt % based on 100 wt % of the total weight of the acidic compound and the alcohol compound.

4. The biodegradable polyester elastomer according to claim 1, wherein the acidic compound is selected from polybasic acids having more than 6 carbon atoms, the alcohol compound is selected from polyhydric alcohols having 4 to 10 carbon atoms, dihydric alcohols having 4 to 10 carbon atoms, and any combination thereof.

5. The biodegradable polyester elastomer according to claim 4, wherein the molar ratio of the alcohol compound to the acidic compound is 2:1 or greater than 2:1.

6. The biodegradable polyester elastomer according to claim 1, wherein the acidic compound is sebacic acid, and the alcohol compound is glycerol.

7. The biodegradable polyester elastomer according to claim 1, wherein the esterification reaction is carried out at a temperature in the range of 120 to 170° C.

8. The biodegradable polyester elastomer according to claim 1, wherein the esterification reaction is carried out at a vacuum pressure in the range of 300 to 500 mTorrs.

9. The biodegradable polyester elastomer according to claim 1, wherein the esterification reaction is carried out under a reaction pressure less than 760 mTorrs.

10. A tissue engineering scaffold material which comprises the biodegradable polyester elastomer according to claim 1.
